SEQUENCE LISTING (17 sequences)

SEQ ID NO: 1 - protein, 196 aa, Artificial Sequence
  FP-151
MAKPSYPPTYKAKPSYPPTYKAKPSYPPTYKAKPSYPPTYKAKPSYPPTYKAKPSYPPTYKSSEEYKGGYYPGNTYHYHSGGSYHGSGYHGGYKGKYYGKAKKYYYKYKNSGKYKYLKKARKYHRKGYKKYYGGSSAKPSYPPTYKAKPSYPPTYKAKPSYPPTYKAKPSYPPTYKAKPSYPPTYKAKPSYPPTYK

SEQ ID NO: 2 - protein, 202 aa, Artificial Sequence
  FP-151-RGD
MAKPSYPPTYKAKPSYPPTYKAKPSYPPTYKAKPSYPPTYKAKPSYPPTYKAKPSYPPTYKSSEEYKGGYYPGNTYHYHSGGSYHGSGYHGGYKGKYYGKAKKYYYKYKNSGKYKYLKKARKYHRKGYKKYYGGSSAKPSYPPTYKAKPSYPPTYKAKPSYPPTYKAKPSYPPTYKAKPSYPPTYKAKPSYPPTYKGRGDSP

SEQ ID NO: 3 - protein, 172 aa, Artificial Sequence
  FP-131
MAKPSYPPTYKAKPSYPPTYKAKPSYPPTYKAKPSYPPTYKAKPSYPPTYKAKPSYPPTYKPWADYYGPKYGPPRRYGGGNYNRYGRRYGGYKGWNNGWKRGRWGRKYYGSAKPSYPPTYKAKPSYPPTYKAKPSYPPTYKAKPSYPPTYKAKPSYPPTYKAKPSYPPTYKL

SEQ ID NO: 4 - protein, 46 aa, Artificial Sequence
  FP-3
ADYYGPKYGPPRRYGGGNYNRYGRRYGGYKGWNNGWKRGRWGRKYY

SEQ ID NO: 5 - protein, 76 aa, Artificial Sequence
  FP-5
SSEEYKGGYYPGNTYHYHSGGSYHGSGYHGGYKGKYYGKAKKYYYKYKNSGKYKYLKKARKYHRKGYKKYYGGGSS

SEQ ID NO: 6 - protein, 10 aa, Artificial Sequence
  fragment sequence derived from FP-1
AKPSYPPTYK

SEQ ID NO: 7 - protein, 60 aa, Artificial Sequence
  FP-1
AKPSYPPTYKAKPSYPPTYKAKPSYPPTYKAKPSYPPTYKAKPSYPPTYKAKPSYPPTYK

SEQ ID NO: 8 - protein, 3 aa, Artificial Sequence
  RGD Group 1
RGD

SEQ ID NO: 9 - protein, 4 aa, Artificial Sequence
  RGD Group 2
RGDS

SEQ ID NO: 10 - protein, 4 aa, Artificial Sequence
  RGD Group 3
RGDC

SEQ ID NO: 11 - protein, 4 aa, Artificial Sequence
  RGD Group 4
RGDV

SEQ ID NO: 12 - protein, 10 aa, Artificial Sequence
  RGD Group 5
RGDSPASSKP

SEQ ID NO: 13 - protein, 5 aa, Artificial Sequence
  RGD Group 6
GRGDS

SEQ ID NO: 14 - protein, 6 aa, Artificial Sequence
  RGD Group 7
GRGDTP

SEQ ID NO: 15 - protein, 6 aa, Artificial Sequence
  RGD Group 8
GRGDSP

SEQ ID NO: 16 - protein, 7 aa, Artificial Sequence
  RGD Group 9
GRGDSPC

SEQ ID NO: 17 - protein, 5 aa, Artificial Sequence
  RGD Group 10
YRGDS

(19) United States
(12) Patent Application Publication
(10) Pub. No.: US 2013/0052712 A1
(43) Pub. Date: Feb. 28, 2013

(54) SURFACE IMMOBILIZATION OF VARIOUS FUNCTIONAL BIOMOLECULES USING MUSSEL ADHESIVE PROTEIN

(71) Applicants: Hyung Joon Cha, Pohang (KR); Bum Jin Kim, Gwangju (KR); Yoo Seong Choi, Seoul (KR); Bong-Hyuk Choi, Daejeon (KR)

(72) Inventors: Hyung Joon Cha, Pohang (KR); Bum Jin Kim, Gwangju (KR); Yoo Seong Choi, Seoul (KR); Bong-Hyuk Choi, Daejeon (KR)

(73) Assignee: POSTECH ACADEMY-INDUSTRY FOUNDATION, Pohang (KR)

(21) Appl. No.: 13/429,610

(22) Filed: Mar. 26, 2012

(30) Foreign Application Priority Data
Aug. 24, 2011 (KR) 10-2011-0084543

(51) Int. Cl. C12N 11/02; B05D 3/00; C12N 5/071; B82Y 5/00

(52) U.S. Cl. 435/177; 435/395; 435/398; 427/2.31; 977/762

(57) ABSTRACT

The present invention relates to technology of immobilizing or coating various functional bioactive substances on various surfaces without physical chemical treatment using mussel adhesive protein. More specifically, the present invention relates to a functional scaffold for tissue engineering comprising artificial extracellular matrix, manufactured by coating various functional bioactive substances on the surface of nanofiber and metal scaffold using mussel adhesive protein, and a method of manufacturing the same.

### CROSS REFERENCE TO RELATED APPLICATION

This application claims priority to and the benefit of Korean Patent Application No. 10-2011-0084543 filed on Aug. 24, 2011, which is hereby incorporated by reference for all purposes as if fully set forth herein.

### BACKGROUND OF THE INVENTION

(a) Field of the invention

The present invention relates to technology of immobilizing or coating various functional bioactive substances on various surfaces without physical chemical treatment using mussel adhesive protein. More specifically, the present invention relates to a scaffold for tissue engineering manufactured by coating various functional bioactive substances on the surface of nanofiber and metal scaffold using mussel adhesive protein, and a method of manufacturing the same.

(b) Description of the Related Art

Tissue engineering technology refers to a technology of culturing cells on a scaffold to prepare a cell-scaffold composite and regenerating biological tissue and organs using the same. Basic principles of tissue engineering technology is gathering required tissue from the body of a patient, isolating cells from the tissue, culturing the isolated cells on a scaffold to prepare a cell-scaffold composite, and then, implanting the prepared cell-scaffold composite into a human body again. Tissue engineering technology is applied for regeneration of most organs of human body including artificial skin, artificial bone, artificial cartilage, artificial cornea, artificial vessel, artificial muscle, and the like.

To optimize regeneration of biological tissue and organs in the tissue engineering technology, it is important to prepare a scaffold similar to biological tissue.

As the basic requirements of the scaffold, tissue cell should be adhered to a scaffold and grows well, the function of differentiated cells should be maintained, the scaffold should be reconciled well with surrounding tissue even after implanted into human body, and it should be non-toxic and biocompatible so that inflammatory reaction or blood coagulation may not occur. And, it should be biodegradable so that it may be completely degraded and disappear in the body within a desired time if implanted cells play a function as new internal tissue.

Currently used polymer scaffolds include natural polymer such as collagen, chitosan, gelatin, hyaluronic acid, alginic acid, and the like, and synthetic polymer such as polylactic acid (PLA), polyglycolic acid (PGA), polycaprolactic acid (PCL), and a copolymer thereof, and the scaffold has been introduced as a scaffold for culture and implant of cells or tissue, cosmetics, medical material such as wound dressing, dental matrix, and the like. However, since they have limited properties, they have many limitations as a material for regeneration of tissue and organs of human body, which requires various properties. And, there has been great difficulty in sufficiently attaching, maintaining and growing bioactive substance such as cells on the surface of the scaffold. Therefore, there is a need for development of a scaffold for tissue engineering that may replace the existing material.

Meanwhile, marine organisms of mussel may securely attach itself to the solid surface such as a rock in the sea by producing and secreting adhesive proteins, and thus, it is not affected by impact of wave or buoyancy of seawater. Mussel adhesive protein is known as strong natural adhesive, and it exhibits about two times higher tensile strength than most epoxy resins, compared to chemical synthetic adhesive, while having flexibility. And, mussel adhesive protein may be adhered to various surfaces such as plastic, glass, metal, Teflon, and biomaterial, and the like, and it may be also adhered to wet surface, which is a problem that has yet to be fully solved, within a few minutes. The inventors found out that since mussel adhesive protein does not attack human cells or cause immune reactions, it is most likely to be applied in the medical fields such as adhesion of biotissue at surgery or adhesion of broken tooth, and the like, and furthermore, it may be used for surface adhesion of cells, and thus, it may be applied in the field of cell culture and tissue engineering.

However, since about ten thousand of mussels are required to obtain 1 g of adhesive material naturally extracted from mussel, although mussel adhesive protein has very excellent properties, there are many limitations in industrially utilizing naturally extracted mussel adhesive protein. As one alternative, mass production of mussel adhesive proteins using recombination of genes have been conducted on Mefp(Mytilus edulis foot protein)-1, Mgfp(Mytilus galloprovincialis foot protein)-1, Mcfp(Mytilus coruscus foot protein)-1, Mefp-2, Mefp-3, Mgfp-3 and Mgfp-5, and the like, which is still insufficient for obtaining significant production amount.

In the previous study, the inventors developed new form of mussel adhesive protein fp-151, wherein decapeptide that is repeated about 80 times in Mefp-1 is 6 time repeatedly fused at both ends of Mgfp-5, and identified that the recombinant mussel adhesive protein may be mass produced in E. coli, and the purification process is very simple, and thus, the industrial applicability is very high (WO2006/107183 or WO2005/092920).

The inventors constructed a two-dimensional surface and a three-dimensional scaffold including mussel adhesive protein to use the mussel adhesive protein obtained by previous studies for tissue engineering, and effectively coated various functional materials on the two-dimensional surface and three-dimensional scaffold without separate physical chemical treatment, thus confirming that it may be provided as a scaffold for tissue engineering including artificial extracellular matrix and medical material, and completed the invention.

### SUMMARY OF THE INVENTION

It is the object of the present invention to provide a scaffold for tissue engineering comprising mussel adhesive protein and bioactive substance attached to the mussel adhesive protein.

Specifically, the scaffold for tissue engineering is a nanofiber scaffold for tissue engineering, comprising nanofiber comprising mussel adhesive protein or mussel adhesive protein and biodegradable polymer, and bioactive substance coated on the surface of the nanofiber.

And, the scaffold for tissue engineering comprises a metal surface coated with the mussel adhesive protein, and bioactive substance coated on the metal surface.

It is another object of the present invention to provide a method for manufacturing the scaffold for tissue engineering comprising mussel adhesive protein.

### BRIEF DESCRIPTION OF THE DRAWINGS

FIG. 1 shows the result of confirming that mussel adhesive protein and hyaluronic acid are coated on the titanium surface through water contact angle and immunostaining. NC represents negative control, MAP represents mussel adhesive protein fp-151, and MAP/HA represents mussel adhesive protein/hyaluronic acid coating.

FIG. 2 shows the result of confirming that mussel adhesive protein and hyaluronic acid are coated on the titanium surface through X-ray photoelectron spectroscopy. NC represents negative control, HA represents hyaluronic acid, MAP represents mussel adhesive protein fp-151, and MAP/HA represents mussel adhesive protein/hyaluronic acid coating.

FIG. 3 shows the result of confirming that mussel adhesive protein and various functional bioactive substances are coated on the titanium surface through Alcian blue staining. NC represents negative control, MAP represents mussel adhesive protein fp-151, MAP/HA represents mussel adhesive protein/hyaluronic acid coating, MAP/HS represents mussel adhesive protein/heparin sulfate coating, MAP/CS represents mussel adhesive protein/chondroitin sulfate coating, and MAP/DS represents mussel adhesive protein/dermatan sulfate coating.

FIG. 4 shows attachment and growth of osteoblast on the titanium surface coated with mussel adhesive protein and hyaluronic acid. NC represents negative control, HA represents hyaluronic acid, MAP represents mussel adhesive protein fp-151, and MAP/HA represents mussel adhesive protein/hyaluronic acid coating.

FIG. 5 shows spreading of osteoblast on the surface coated with mussel adhesive protein and hyaluronic acid. NC represents negative control, HA represents hyaluronic acid, MAP represents mussel adhesive protein fp-151, and MAP/HA represents mussel adhesive protein/hyaluronic acid coating.

FIG. 6 shows differentiation of osteoblast on the titanium surface coated with mussel adhesive protein and hyaluronic acid. NC represents negative control, HA represents hyaluronic acid, MAP represents mussel adhesive protein fp-151, and MAP/HA represents mussel adhesive protein/hyaluronic acid coating.

FIG. 7 shows nanofiber prepared through blending of various synthetic polymers (PCL, PDO, PLLA, PLGA, PEO, PVA) and mussel adhesive protein.

FIG. 8 shows electron microscope images of nanofibers prepared by blending PCL and mussel adhesive protein at various ratios.

FIG. 9 shows the results of analyzing contact angles of PCL, PCL/mussel adhesive protein nanofibers.

FIG. 10 shows the analysis results of Fourier-transform infrared spectroscopy of PCL, PCL/mussel adhesive protein nanofibers.

FIG. 11 shows the analysis results of X-ray photoelectron spectroscopy of PCL, PCL/mussel adhesive protein nanofibers.

FIG. 12 shows stress-strain curves of PCL, PCL/mussel adhesive protein nanofibers.

FIG. 13 shows measurement values of mechanical properties of PCL, PCL/mussel adhesive protein nanofibers.

FIG. 14 shows electron microscope image of PCL/mussel adhesive protein nanofiber.

FIG. 15 shows the analysis results of the shape and population of osteoblast on PCL, PCL/mussel adhesive protein nanofibers.

FIG. 16 shows the analysis results of osteoblast attachment and growth degrees on PCL, PCL/mussel adhesive protein nanofibers.

FIG. 17 shows the images of green fluorescent protein coating on PCL, PCL/mussel adhesive protein nanofibers.

FIG. 18 shows the images of nucleic acid coating on PCL, PCL/mussel adhesive protein nanofibers.

FIG. 19 shows the images of hyaluronic acid coating on PCL, PCL/mussel adhesive protein nanofibers.

FIG. 20 shows the images of coating of various saccharides, hyaluronic acid (HA), heparin sulfate (HS), chondroitin sulfate (CS) and natural saccharide alginate (AG) on PCL, PCL/mussel adhesive protein nanofibers.

FIG. 21 shows the activities of alkaline phosphatase coated on PCL, PCL/mussel adhesive protein nanofibers.

FIG. 22 shows the images of antibody coating on PVA, PVA/mussel adhesive protein nanofibers.

### DETAILED DESCRIPTION OF THE INVENTION

To solve the objects of the invention, one aspect of the invention provides a scaffold for tissue engineering comprising mussel adhesive protein and bioactive substance attached to the mussel adhesive protein.

The mussel adhesive protein is adhesive protein originated from mussel, preferably Mytilus edulis, Mytilus alloprovincialis or Mytilus coruscus or a variant thereof, but not limited thereto. For example, the mussel adhesive protein may include fp(foot protein)-1 to fp-5 protein respectively originated from the mussel species or a variant thereof, preferably Mefp(Mytilus edulis foot protein)-1, Mgfp(Mytilus galloprovincialis foot protein)-1, Mcfp(Mytilus coruscus foot protein)-1, Mefp-2, Mefp-3, Mgfp-3 and Mgfp-5 or a variant thereof, but not limited thereto.

And, the mussel adhesive protein may include all mussel adhesive protein described in WO2006/107183 or WO2005/092920. Preferably, the mussel adhesive protein may include Mgfp-3 protein consisting of amino acid sequence set forth in SEQ ID NO. 4, Mgfp-5 protein consisting of amino acid sequence set forth in SEQ ID NO. 5, or a variant thereof, but not limited thereto. The mussel adhesive protein may include polypeptide wherein fp-1 protein consisting of amino acid sequence set forth in SEQ ID NO. 6 is 1 to 10 times consecutively connected. And, the mussel adhesive protein may include a fusion polypeptide of at least two kinds selected from the group consisting of SEQ ID NO. 4, SEQ ID NO. 5, and consecutive polypeptide of SEQ ID NO. 6, and preferably, the fusion polypeptide may include fp-151 protein set forth in SEQ ID NO. 1 or fp-131 protein set forth in SEQ ID NO. 3, but not limited thereto.

The variant of mussel adhesive protein may preferably include additional sequence at C-terminal or N-terminal of mussel adhesive protein, as long as it maintains adhesion of mussel adhesive protein, or some amino acids may be substituted with other amino acids. More preferably, it may be those wherein polypeptide consisting of 3 to 25 amino acids comprising RGD (Arg Gly Asp) is connected to the C-terminal or N-terminal of the mussel adhesive protein, or 1 to 100%, preferably 5 to 100% of the total tyrosine residues constituting the mussel adhesive protein are substituted with 3,4-dihydroxyphenyl-L-alanine (DOPA).

The 3 to 25 amino acids comprising RGD may be preferably selected from the group consisting of RGD(Arg Gly Asp, SEQ ID NO. 8), RGDS(Arg Gly Asp Ser, SEQ ID NO. 9), RGDC(Arg Gly Asp Cys, SEQ ID NO. 10), RGDV(Arg Gly Asp Val, SEQ ID NO. 11), RGDSPASSKP(Arg Gly Asp Ser Pro Ala Ser Ser Lys Pro, SEQ ID NO. 12), GRGDS(Gly Arg Gly Asp Ser, SEQ ID NO. 13), GRGDTP(Gly Arg Gly Asp Thr Pro, SEQ ID NO. 14), GRGDSP(Gly Arg Gly Asp Ser Pro, SEQ ID NO. 15), GRGDSPC(Gly Arg Gly Asp Ser Pro Cys, SEQ ID NO. 16), YRGDS(Tyr Arg Gly Asp Ser, SEQ ID NO. 17), and a combination thereof, but not limited thereto.

The variant of mussel adhesive protein wherein polypeptide consisting of 3 to 25 amino acids comprising RGD is connected to C-terminal or N-terminal of the mussel adhesive protein may be preferably fp-151-RGD polypeptide consisting of amino acid sequence set forth in SEQ ID NO. 2, but not limited thereto.

The bioactive substance attached to the mussel adhesive protein generally refers to substances involved in actions of promoting cell growth and differentiation through interactions with cells or tissues of human body, and assisting in tissue regeneration and recovery. Also, the bioactive substance generally refers to various biomolecules that may be included to embody an artificial extracellular matrix with a similar structure to a natural extracellular matrix.

According to the present invention, various kinds of functional bioactive substances may be conveniently coated without physical chemical treatment using mussel adhesive protein. The bioactive substance may include cell, protein, nucleic acid, fatty acid, carbohydrate, enzyme, antibody, and the like. For example, it may be the bioactive substance is selected from the group consisting of osteoblast, fibroblast, hepatocyte, neuron, cancer cell, B cell, white blood cell, stem cell, hyaluronic acid, heparan sulfate, chondroitin sulfate, alginate, dermatan sulfate, alkaline phosphatase, DNA, RNA, stem cell factor (SCF), vascular endothelial growth factor (VEGF), transforming growth factor (TGF), fibroblast growth factor (FGF), epidermal growth factor (EGF), cartilage growth factor (CGF), nerve growth factor (NGF), keratinocyte growth factor (KGF), skeletal growth factor (SGF), osteoblast-derived growth factor (BDGF), hepatocyte growth factor (HGF), insulin-like growth factor (IGF), cytokine growth factor (CGF), platelet-derived growth factor (PDGF), epithelial growth factor (EGF), bone growth factor, placental growth factor (PlGF), heparin-binding epidermal growth factor (HB-EGF), endothelial cell growth supplement (EGGS), colony stimulating factor (CSF), granulocyte macrophage-colony stimulating factor (GM-CSF), granulocyte colony stimulating factor (G-CSF), growth differentiation factor (GDF), integrin modulating factor (IMF), calmodulin (CaM), thymidinc kinase (TK), tumor necrosis factor (TNF), growth hormones (GH), growth hormone releasing hormone, growth hormone releasing peptide, glucagon-like peptides, G-protein-coupled receptor, macrophage activating factor, erythropoietin, macrophage peptide, B cell factor, T cell factor, protein A, allergy inhibitor, immunotoxin, lymphotoxin, tumor necrosis factor, tumor suppressors, metastasis growth factor, alpha-1 antitrypsin, albumin, alpha-lactalbumin, apolipoprotein-E, angiopoietins, hemoglobin, thrombin, thrombin receptor activating peptide, thrombomodulin, factor VII, factor VIIa, factor VIII, factor IX, factor XIII, plasminogen activating factor, fibrin-binding peptide, urokinase, streptokinase, hirudin, protein C, C-reactive protein, renin inhibitor, collagenase inhibitor, superoxide dismutase, leptin, angiostatin, angiotensin, bone stimulating protein, calcitonin, insulin, atriopeptin, cartilage inducing factor, elcatonin, connective tissue activating factor, tissue factor pathway inhibitor, follicle stimulating hormone, luteinizing hormone, luteinizing hormone releasing hormone, parathyroid hormone, relaxin, secretin, somatomedin, adrenocortical hormone, glucagon, cholecystokinin, pancreatic polypeptide, gastrin releasing peptide, corticotropin releasing factor, thyroid stimulating hormone, autotaxin, lactoferrin, myostatin, receptors, receptor antagonists, cell surface antigens, virus derived vaccine antigens, bone morphogenetic proteins (BMP), matrix metalloproteinase (MMP), tissue inhibitor matrix metalloproteinase (TIMP), interferons, interferon receptors, interleukins, interleukin receptors, interleukin binding proteins, cytokines, cytokine binding proteins, integrins, selectins, cadherins, collagen, elastin, lectins, fibrillins, nectins, fibronectin, vitronectin, hemonectin, laminin, glycosaminoglycans, hemonectin, thrombospondin, heparan sulfate, vitronectin, proteoglycans, transferrin, cytotactin, tenascin, lymphokines, neural cell adhesion molecules (N-CAMS), intercellular cell adhesion molecules (ICAMS), vascular cell adhesion molecule (VCAM), platelet-endothelial cell adhesion molecule (PECAM), monoclonal antibodies, polyclonal antibodies, antibody fragments, and combinations thereof, but not limited thereto.

The present invention provides a scaffold for tissue engineering.

Tissue engineering technology refers to technology of culturing cells isolated from the tissue of a patient on a scaffold to prepare a cell-scaffold composite, and then, implanting the cell-scaffold composite into a human body, and it is applied for regeneration of most organs of human body including artificial skin, artificial bone, artificial joint, artificial cornea, artificial vessel, artificial muscle, and the like. According to the present invention, a scaffold similar to biotissue may be provided to optimize regeneration of biotissue and organs in tissue engineering technology. And, the scaffold of the present invention may be used to conveniently embody an artificial extracellular matrix, and it may be utilized as cosmetics, medical material such as wound dressing, dental matrix, and the like.

According to one preferable embodiment, the invention provides a nanofiber scaffold for tissue engineering, comprising nanofiber comprising mussel adhesive protein or mussel adhesive protein and biodegradable polymer, and bioactive substance coated on the surface of the nanofiber.

According to another embodiment, the present invention provides a method for manufacturing a scaffold for tissue engineering, comprising:

(1) preparing a nanofiber scaffold from mussel adhesive protein alone, or by mixing mussel adhesive protein and biodegradable polymer; and

(2) coating bioactive substance on the surface of the nanofiber scaffold.

In the step (1), a nanofiber scaffold is prepared from mussel adhesive protein alone, or by mixing mussel adhesive protein and biodegradable polymer. A natural extracellular matrix consists of a three-dimensional structure wherein nanosized protein fibers are entangled. Thus, the present invention utilizes nanofiber to embody a similar structure to natural extracellular matrix. Since the nanofiber has a large cell adhesion area due to the large specific surface area, if cells are cultured on a scaffold consisting of nanofibers, cell adhesion may become excellent.

To prepare the three-dimensional nanofiber scaffold, it is preferable to use an electrospinning process.

The electrospinning process is a technology of forming fiber using attractive force and repulsive force generated when a polymer solution or molten polymer is charged to a predetermined voltage. According to the electrospinning process, fibers with various diameters including several nm to several hundred nm may be prepared, the structure of the equipment is simple, it may be applied for various materials, and porosity may be increased compared to existing fibers, thus enabling preparation of fiber having large surface area to volume ratio.

Specifically, to perform the electrospinning process, first, mussel adhesive protein may be dissolved in an organic solvent alone or in a mixed solvent of an organic solvent and an acid solvent. The acid solvent may include phosphoric acid, acetic acid, formic acid, hydrochloric acid, sulfuric acid, nitric acid, and the like, but not limited thereto, and acetic acid is preferable. The organic solvent may include HFIP (hexafluoroisopropanol), HFP (hexafluoropropanol), TFA (trifluoroacetic acid), and the like, but not limited thereto, and HFIP is preferable. The mixing ratio of the organic solvent and the acidic solvent may include various ratios where the mussel adhesive protein may not be precipitated and be appropriately dissolved, and it may be preferable to mix HFIP and acetic acid in the ratio of 90:10 (v/v). The concentration of the mussel adhesive protein dissolved in the solvent may be in the range of 10˜15% (w/v), preferably 12% (w/v). The electrospinning process may be performed using appropriate spinning equipment while applying appropriate voltage. It may be preferable to apply voltage in the range of 10 to 20 kV at electrospinning because stable electrospinning may be conducted. As voltage increases in the range of 10 to 20 kV, electrospinning speed increases.

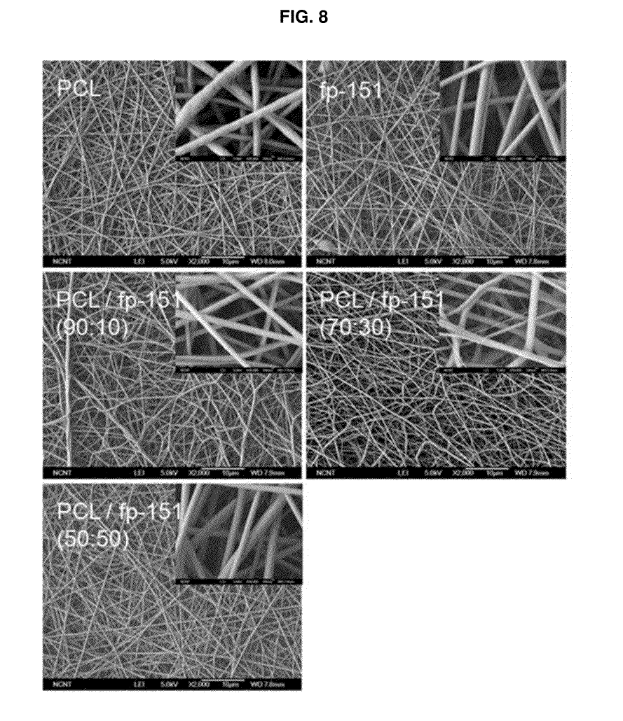

Specific example of the invention shows that nanofiber may be successfully prepared by electrospinning only with a mussel adhesive protein solution under the above conditions (FIG. 8).

Although nanofiber may be prepared by dissolving mussel adhesive protein alone in an organic solvent, and the like, and then, conducting electrospinning, it may be prepared by blending mussel adhesive protein with biodegradable polymer to prepare a synthetic polymer solution, and then, conducting electrospinning.

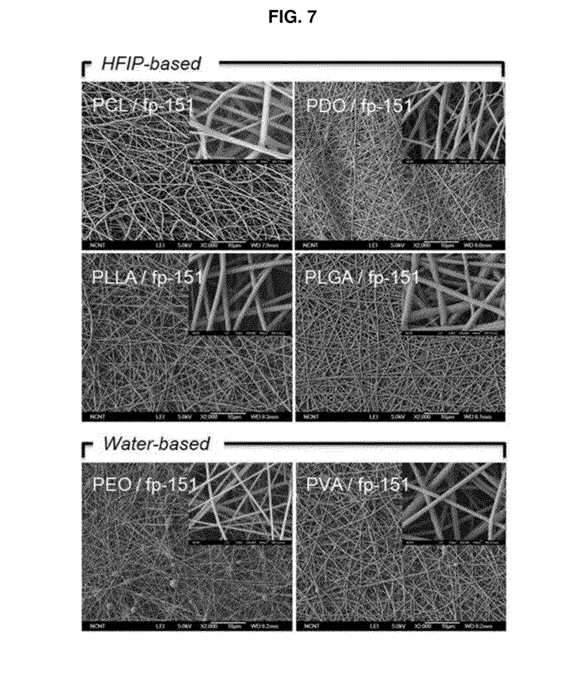

The polymer may include most biodegradable polymers generally used as tissue engineering material. In the present invention, PCL (polycaprolactone), PDO (polydioxanone), PLLA (poly(L-lactide)) and PLGA (poly(DL-lactide-co-glycolide)), known to be dissolved in a HFIP solvent and well achieve electrospinning, and PEO (polyethylene oxide) and PVA (polyvinyl alcohol), known to be water-soluble, but not limited thereto. PCL, PDO, PLLA and PLGA polymers may be respectively dissolved in a HFIP solvent, and then, blended with the above-explained mussel adhesive protein solution to conduct electrospinning, and PEO and PVA polymers may be respectively dissolved in water, and then, blended with water-dissolved mussel adhesive protein solution to conduct electrospinning. Specific example of the invention shows that nanofiber may be successfully prepared by blending mussel adhesive protein with various kinds of polymer partners under the above conditions, and then, conducting electrospinning (FIG. 7).

Furthermore, the mussel adhesive protein and biodegradable polymer may be mixed at various ratios. According to specific examples, PCL polymer is selected as a blending partner, and PCL and mussel adhesive protein is mixed respectively at a ratio of 90:10, 70:30, and 50:50 (w/w), and then, electrospinning is conducted to prepare nanofiber (FIG. 8).

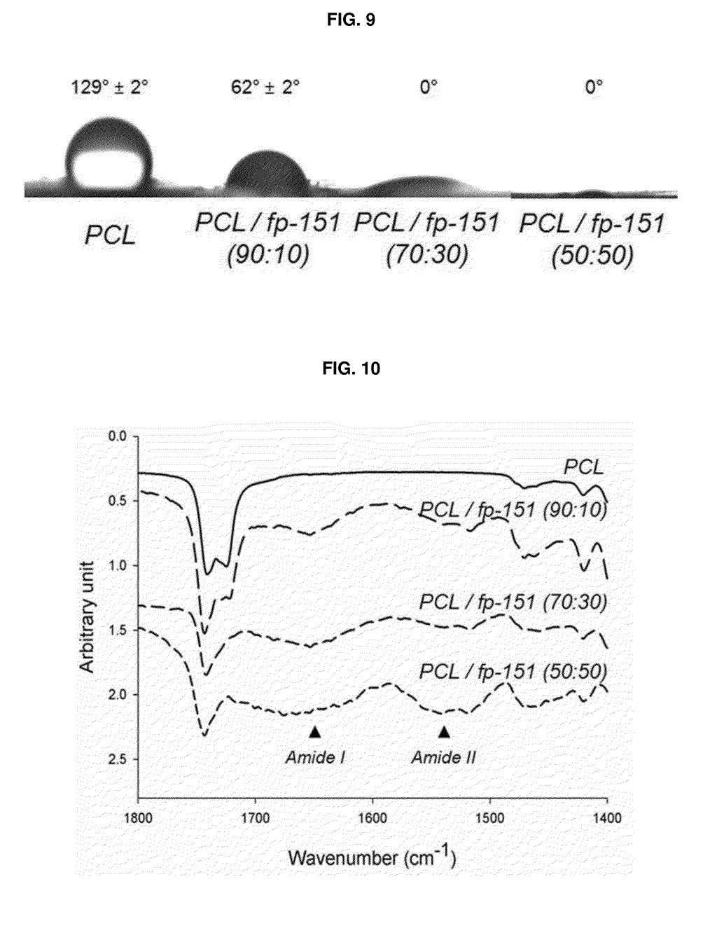

On the surface of the nanofiber prepared by blending mussel adhesive protein and PCL polymer, mussel adhesive protein may be naturally exposed. According to specific examples of the invention, as results of analyzing the nanofiber prepared with PCL and mussel adhesive protein through water contact angle, Fourier-transform infrared spectroscopy, and X-ray photoelectron spectroscopy, it is confirmed that mussel adhesive protein is appropriately exposed on the surface of nanofiber thus providing hydrophilicity (FIGS. 9 to 11).

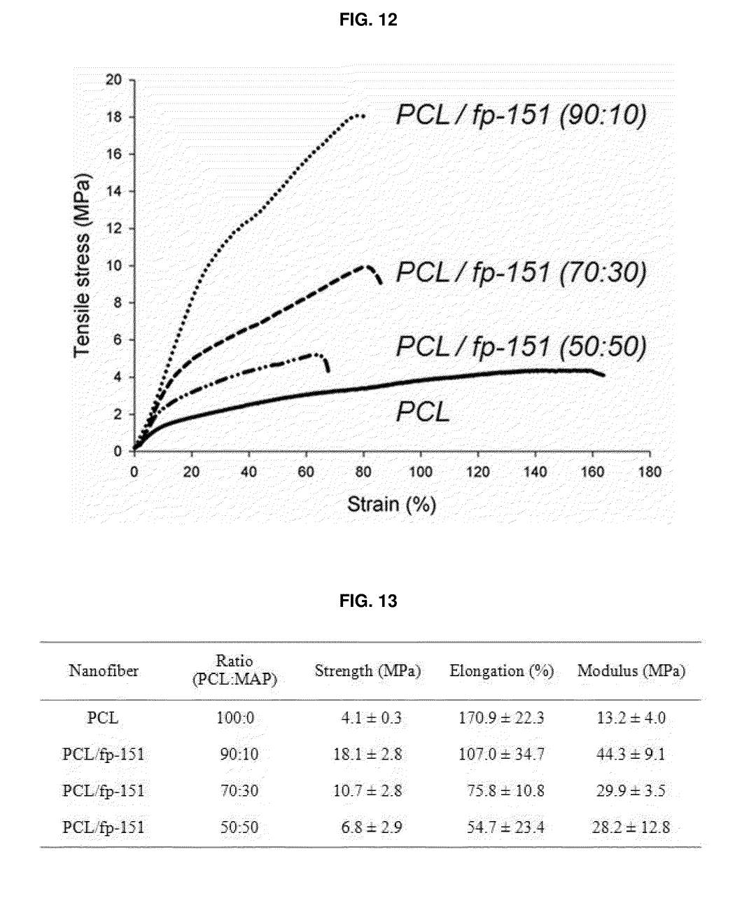

And, the nanofiber prepared with PCL and mussel adhesive protein exhibits excellent tensile strength and Young's modulus. It has been reported that when culturing cells on a scaffold, if mechanical stimulation is given through the scaffold, cells may be made more robust thus favorable for tissue regeneration (Kim et al., Nature Biotechnology, 17:979-983, 1999). According to specific examples of the invention, as results of measuring mechanical properties of the above prepared PCL/mussel adhesive protein nanofibers of various ratios, it is confirmed that they have maximum 4 times of tensile strength and higher Young's modulus, compared to PCL nanofiber (FIGS. 12 and 13). Thus, the nanofiber scaffold of the present invention is expected to exhibit excellent tissue regeneration effect as a biodegradable scaffold having flexibility and elasticity.

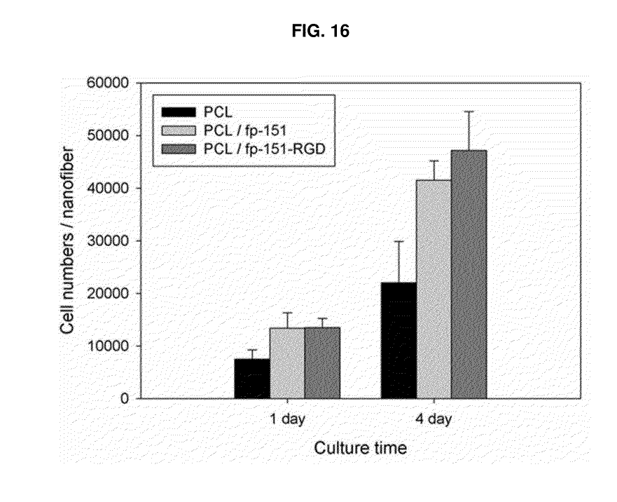

In practice, nanofiber prepared by blending of mussel adhesive protein and PCL polymer exhibited good interactions with cells. According to specific examples, as results of culturing osteoblast (MC3T3-E1) on the above prepared PCL/mussel adhesive protein nanofiber in order to evaluate cell culture performance of the nanofiber scaffold of the present invention, it was confirmed that the cells well spread along the fiber, and the degrees of adhesion and growth of the cells are improved (FIGS. 15 and 16).

In the step (2), various functional bioactive substances are coated on the surface of the above prepared three-dimensional nanofiber scaffold.

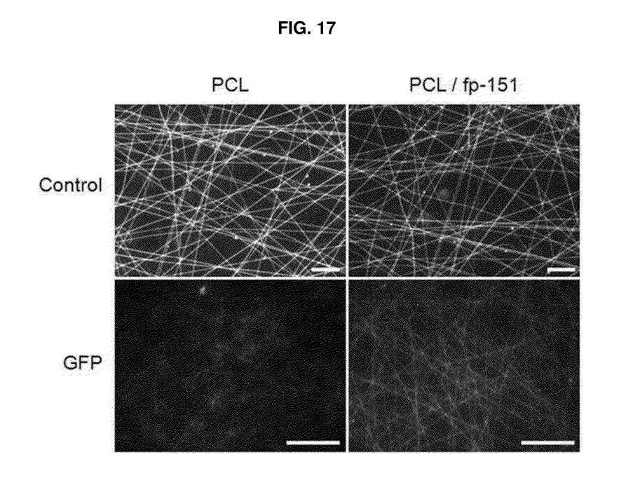

According to the present invention, various kinds of functional bioactive substances including protein, nucleic acid, saccharide, enzyme, and the like may be conveniently coated on the surface of the above prepared mussel adhesive protein-based three-dimensional nanofiber scaffold without physical chemical treatment. And, various bioactive substances may be coated on the nanofiber scaffold to form an extracellular matrix similar to natural extracellular matrix. According to specific examples, it was confirmed that by coating a solution including various bioactive substance on the surface of a scaffold using the above prepared PCL/mussel adhesive protein (70:30) nanofiber, corresponding substances may be uniformly coated simply along the nanofiber surface (FIGS. 17 to 21).

From the above results it can be seen that a nanofiber scaffold using mussel adhesive protein may be applied for a carrier for tissue engineering and medical material.

According to yet another embodiment, the present invention provides a scaffold for tissue engineering comprising a metal surface coated with mussel adhesive protein, and bioactive substance coated on the metal surface.

According to yet another embodiment, the present invention provides a method for manufacturing the above scaffold for tissue engineering, comprising

(1) coating mussel adhesive protein on a metal surface; and

(2) coating bioactive substance on the metal surface.

The step (1) is a step of coating a two-dimensional surface using mussel adhesive protein, wherein dip coating may be conveniently conducted by immersing the two-dimensional surface in a solution including mussel adhesive protein. The two-dimensional surface to be coated with mussel adhesive protein may include a surface formed of metal or polymer material that can be used for tissue engineering material. According to specific examples, a mussel adhesive protein solution is coated on a titanium surface homogenized with a piranha solution.

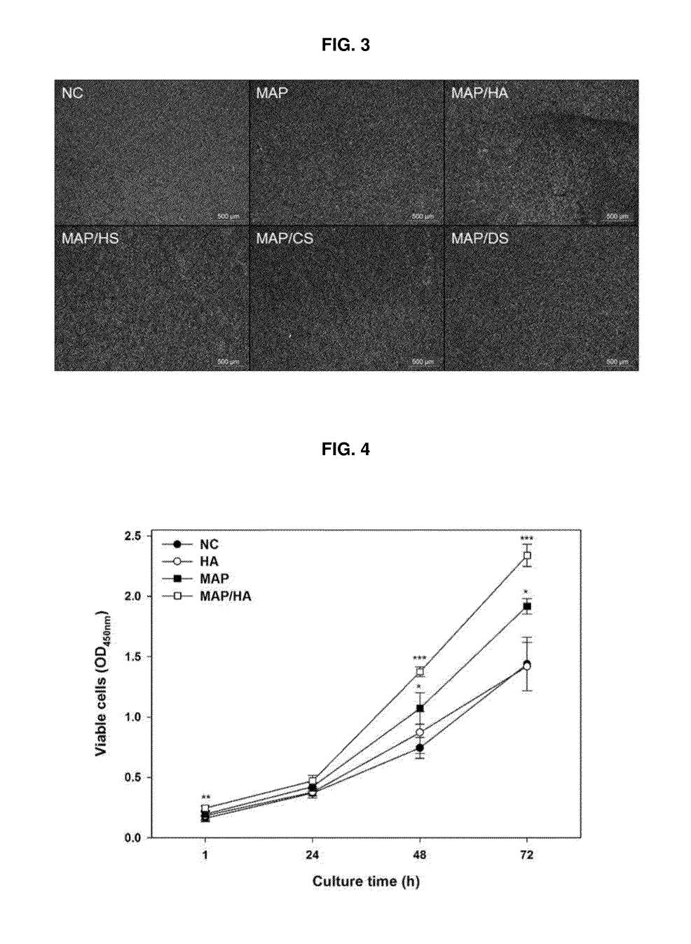

The step (2) is a step of conveniently coating various functional bioactive substances on the surface coated with mussel adhesive protein without physical chemical treatment. According to the present invention, negatively charged various functional bioactive substances may be coated on the metal surface coated with mussel adhesive protein. According to specific examples, hyaluronic acid, heparan sulfate, chondroitin sulfate and dermatan sulfate constituting an extracellular matrix may be effectively coated on the two-dimensional surface coated with mussel adhesive protein, respectively (FIG. 3). The present invention may assist in improvement in cell functionality by coating bioactive material that can interact with cells, as explained.

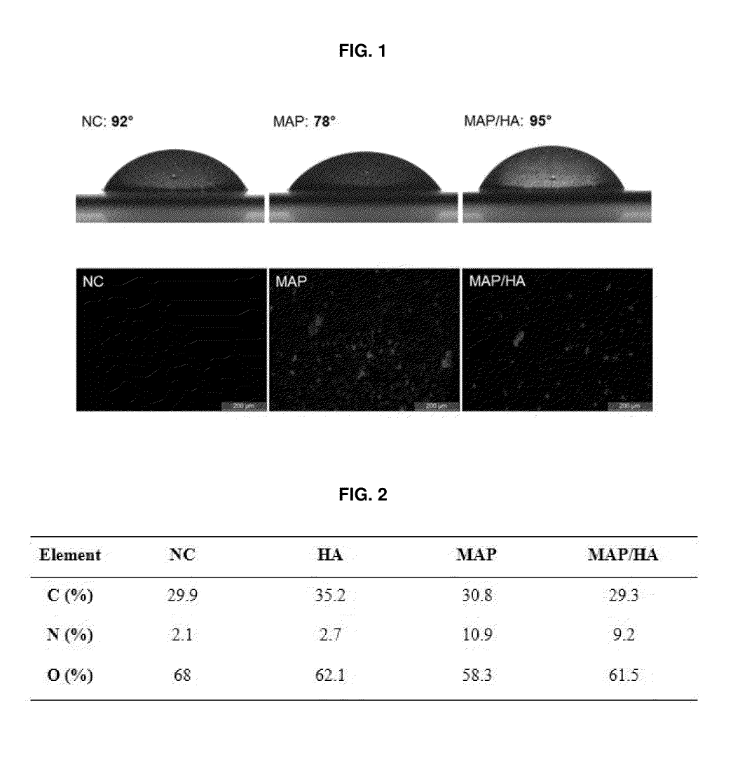

According to specific examples, hyaluronic acid was coated on the titanium surface coated with mussel adhesive protein, and then, successive coating was confirmed by water contact angle, immunostaining, X-ray photoelectron spectroscopy, and Alcian blue staining (FIGS. 1 to 3). And, as results of culturing osteoblast on the above surface and analyzing improvement in cell functionality, it was confirmed that cell adhesion, growth and differentiation degrees are improved by hyaluronic acid coating (FIGS. 4 to 6).

The two-dimensional metal scaffold using mussel adhesive protein may be used for tissue engineering material, for example, scaffold material for growing cells, and it may be applied for equipment or device requiring various polymer multilayers, including biosensor such as cell-based biosensor or chemosensor such as chemical sensor.

According to the present invention, various functional bioactive substances may be coated on a two-dimensional surface or three-dimensional surface without complicated physical chemical pretreatment using adhesion of mussel adhesive protein, and thus, an artificial extracellular matrix may be conveniently embodied, and the present invention may be applied for development of a biofunctional scaffold for tissue engineering.

Hereinafter, the present invention will be explained in detail with reference to the following examples. However, these examples are only to illustrate the invention, and the scope of the invention is not limited thereto.

### Example 1

### Coating of Various Biomaterials Using Mussel Adhesive Protein

1-1. Coating of Hyaluronic Acid on Titanium Surface Using Mussel Adhesive Protein fp-151

Mussel adhesive protein fp-151 (SEQ ID NO.1) is produced in E. coli, after synthesizing fp-1 variant wherein peptide consisting of AKPSYPPTYK is repeatedly connected 6 times in the amino acid sequence of naturally existing mussel adhesive protein fp-1 (Genbank No. Q27409), and inserting Mgfp-5 gene (Genbank No. AAS00463) between two fp-1 variants. The preparation of the mussel adhesive protein fp-151 is as described in WO 2005/092920, which is incorporated herein by reference.

Using the above prepared mussel adhesive protein fp-151, it was confirmed whether hyaluronic acid, biopolymer of negatively charged electrolyte, is coated on a titanium surface. Specifically, mussel adhesive protein fp-151 and hyaluronic acid having a molecular weight of 17 kDa (Lifecore Biomedical; Minnesota) were respectively dissolved in 10 mM sodium chloride buffer (controlled to pH 5.0 with hydrochloric acid) to a concentration of 1 mg/ml, and titanium with purity of 99.5% or more (Alfa Aesar, MA) was prepared with the size of 10 mm×10 mm. Before coating the mussel adhesive protein fp-151, the surface of the titanium flake was homogenized using a piranha solution. First, the fp-151 solution was coated on the titanium surface for 30 minutes, and then, non-coated fp-151 solution was removed with a spin coater (Jaeseong Engineering, Anyang), and the surface was washed with 10 mM sodium chloride buffer. And then, a hyaluronic acid solution was coated on the titanium surface coated with fp-151 for 30 minutes, remaining hyaluronic acid solution was removed by the above method, and then, the surface was washed with a 10 mM sodium chloride solution. The results of fp-151 and hyaluronic acid coating were confirmed through water contact angle, immuostaining, and X-ray photoelectron spectroscopy (XPS), with a non surface-coated titanium surface as negative control (NC). For analysis of water contact angle, water was dropped on the surface, and then, image was obtained using CCD (charge-coupled device) imaging system (Surface and Electro-Optics). For immunostaining, antibody produced in rabbit using amino acid sequence of mussel adhesive protein was reacted with the surface, and then, secondary antibody to rabbit antibody, to which fluorescent material of Texas red is bonded, was reacted to obtain fluorescent image. At this time, before reacting with antibody, the surface was blocked with a 1% BSA solution, and after reacting, it was washed with TTBS buffer. And, for X-ray photoelectron spectroscopy, carbon (C), nitrogen (N), oxygen (O) atom contents were respectively analyzed using ESCALAB 220iXL (VG Scientific) equipment.

As the results, as shown in FIG. 1, it was confirmed that water contact angle decreased on the titanium surface coated with mussel adhesive protein, and that water contact angle increased on the surface coated with hyaluronic acid using mussel adhesive protein.

It was also confirmed by immunostaining using mussel adhesive protein antibody that intensity of fluorescence was detected high on the surface coated with mussel adhesive protein, but intensity of fluorescence was decreased on the surface coated with hyaluronic acid using mussel adhesive protein.

Furthermore, as shown in FIG. 2, as results of confirming surface elemental constituents through X-ray photoelectron spectroscopy, nitrogen existing in biomaterial was scarcely detected on the non-coated titanium surface, nitrogen content increased 8% or more on the surface coated with mussel adhesive protein, compared to the non-coated surface, and nitrogen content decreased about 2% on the surface coated with hyaluronic acid having relatively low nitrogen content.

These results show that mussel adhesive protein and hyaluronic acid were successively coated on the titanium surface.

1-2. Coating of Various Biomaterials on Titanium Surface Using Mussel Adhesive Protein fp-151

A titanium surface was coated with mussel adhesive protein fp-151 by the same method as <Example 1-1>, and coated with heparan sulfate, chondroitin sulfate, dermatan sulfate, and then, stained with 1% alcian blue for 15 minutes, observed by microscope, and the results were shown in FIG. 3.

As shown in FIG. 3, it was confirmed that various bioactive materials are well coated on the titanium surface using fp-151.

1-3. Attachment and Growth of Osteoblast on Titanium Surface Coated with Hyaluronic Acid Using Mussel Adhesive Protein fp-151

On the titanium surface coated with mussel adhesive protein and hyaluronic acid by the method of <Example 1-1>, cell activity was examined.

Specifically, murine osteoblast (MC3T3-E1; Riken cell bank) were cultured in a 37° C. incubator using animal cell culture medium containing 10% FBS (fetal bovine serum; Hyclone) and 1% antibiotic-antimycotic (Hyclone). The cells were stripped off from the cell culture dish and diluted to a concentration of 2×10⁵/ml in the culture medium that does not contain 10% FBS, and coated titanium flakes were put in 12-well cell culture dish, and then, the cells were introduced 1×10⁵ per well and cultured in an incubator for 1 hour. After the culture, to quantify living cells, CCK-8 (cell counting kit-8) analysis was conducted. First, to remove non-attached cells after the culture, the dish was washed with PBS(phosphate buffered saline; Hyclone) and 50 ul of a CCK-8 solution was injected into the well. Since living cells reduce 2-(2-methoxy-4-nitrophenyl)-3-(4-nitrophenyl)-5-(2,4-disulfophenyl)-2H-tetrazolium (WST-8) to water-soluble formazan in mitochondria, CCK-8 reagent was introduced and the cells were additionally cultured for 3 hours, and then, absorbance of formazan dissolved in the media was measured at 450 nm with spectrophotometer. And, to continuously culture, the cells were washed with PBS, 1 ml of the culture media containing 10% FBS was introduced, and the cells were cultured in a 37° C. incubator. The growth of the cells was measured by the same method as attachment, and the results are shown in FIG. 4.

As shown in FIG. 4, it can be seen that cell attachment and growth on the titanium surface that is coated with hyaluronic acid (17 kDa) using mussel adhesive protein fp-151 were more excellent, compared to the titanium surface coated with only fp-151 or only hyaluronic acid.

1-4. Spreading of Osteoblast on Titanium Surface Coated with Hyaluronic Acid Using Mussel Adhesive Protein fp-151

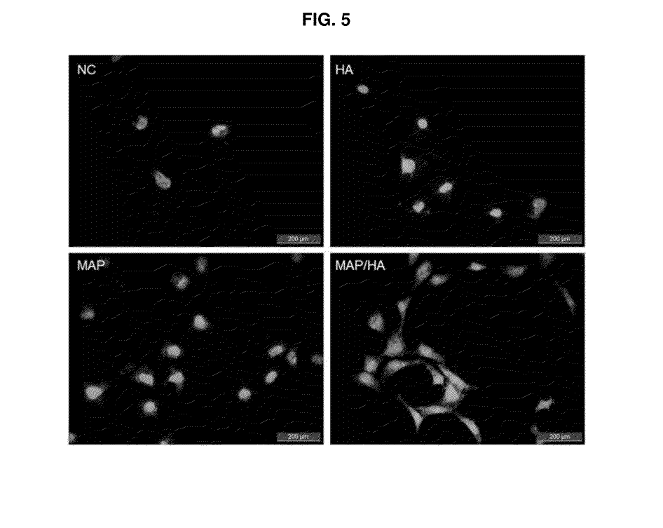

Osteoblast to be cultured by the method of <Example 1-3> and titanium flake were prepared, and the cells were cultured in a culture medium that does not contain 10% FBS for 18 hours. To confirm spreading of the cells, fluorescence staining was conducted using phalloidin to which FITC capable of bonding with actin is conjugated, and confirmed by fluorescence microscopy and shown in FIG. 5.

As shown in FIG. 5, it was confirmed that osteoblast spreads well on the titanium surface coated with hyaluronic acid using fp-151, compared to fp-151 and negative control.

1-5. Differentiation of Osteoblast on Titanium Surface Coated with Hyaluronic Acid (17 kDa) Using Mussel Adhesive Protein fp-151

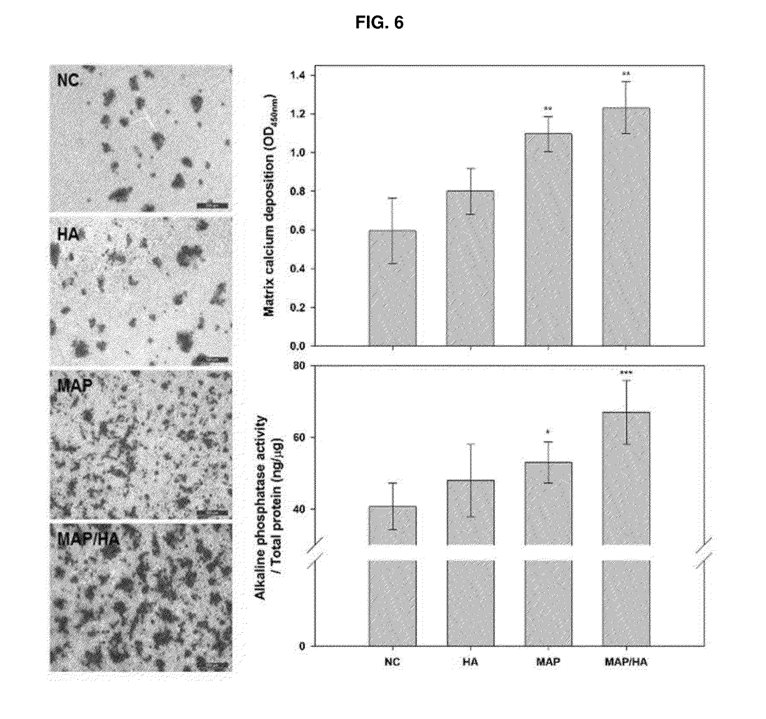

Osteoblast were cultured on a titanium flake by the method of <Example 1-3>, and 50 ug/ml vitamin C (ascorbic acid) and 10 mM sodium phosphate monobasic were added to the culture medium when about 90% of the flake was covered with grown cells to prepare a culture medium for differentiation, which was then treated on the cells for 15 days. To confirm differentiation of the cells, the cells were stained with 2% Alizarin red S for 5 minutes and observed with microscope, and activity of enzymes related to intracellular differentiation was confirmed using an activity examining kit and shown in FIG. 6.

As shown in FIG. 6, it was confirmed that differentiation of osteoblast occurred most on the titanium surface coated with hyaluronic acid using fp-151, compared to fp-151 and negative control.

### Example 2

### Preparation of Mussel Adhesive Protein-Based Nanofiber

2-1. Preparation of Nanofiber Through Blending of Mussel Adhesive Protein fp-151 and Various Synthetic Polymers

Nanofiber was prepared using recombinant mussel adhesive protein fp-151 alone or in combination with PCL (polycaprolactone), PDO (polydioxanone), PLLA (poly(L-lactide)), PLGA (poly(DL-lactide-co-glycolide)), PEO (polyethylene oxide) or PVA (polyvinyl alcohol), which is biodegradable polymer commonly used as tissue engineering material, by electrospinning. For blending with PCL, PDO, PLLA, PLGA, a HFIP (1,1,1,3,3,3-hexafluoroisopropanol)-based solvent was used, and for blending with PEO, PVA, a water-based solvent was used. PCL or PDO was dissolved in HFIP to a concentration of 6% (w/v), and PLLA or PLGA to a concentration of 12% (w/v), and then, fp-151 was dissolved in a solvent containing 90:10 (v/v) of HFIP and acetic acid to a concentration of 12% (w/v). And PCL or PDO was mixed with fp-151 at the ratio of 70:30 (w/w), and PLLA or PLGA was mixed with fp-151 at the ratio of 90:10 (w/w), and electrospining was conducted. PEO or PVA was dissolved in water to a concentration of 6% (w/v), fp-151 was dissolved in water to a concentration of 12% (w/v), they are mixed at the ratio of 70:30 (w/w), and electrospinning was conducted. By electrospinning, the mixture was discharged at a speed of 1 ml/h using a syringe pump, and 15 kV of high voltage was applied when the mixture passes through a needle having a diameter of 0.5 mm, thus producing nanofiber, and the produced nanofiber was received on an aluminum foil located 15 cm away from the needle. As the results, it was confirmed that nanofibers of straight fiber shape are successfully produced by all kinds of synthetic polymers and electrospinning, which are shown in FIG. 7.

2-2. Preparation of Nanofiber by Blending of Mussel Adhesive Protein fp-151 and PCL

PCL was selected as a synthetic polymer partner to blend with fp-151 at various ratios to prepare nanofibers and measure the properties. Since PCL has a longer alkyl chain (5 per monomer) compared to the above used PDO, PLLA, PLGA, and high flexibility and low rigidity, it is expected to have appropriate mechanical properties when forming a complex with protein such as mussel adhesive protein. PCL and fp-151 were dissolved with the same concentration and solvent as the above, and then, they were mixed respectively at the ratio of 100:0, 70:30, 50:50, 0:100 (w/w) to prepare nanofibers with various mixing ratios. It was confirmed that nanofibers are successfully prepared under the same electrospinning conditions as <Example 2-1> and at all ratios, which are shown in FIG. 8.

### Example 3

### Measurement of Properties of Mussel Adhesive Protein-Based Nanofiber

3-1. Surface Hydrophilicity Analysis Through Water Contact Angle

To examine surface hydrophilicities of nanofibers prepared by blending of PCL and fp-151 in <Example 2-2>, i.e., PCL, PCL/fp-151 (90:10), PCL/fp-151 (70:30), PCL/fp-151 (50:50), contact angle was measured. As results of measuring with CCD (charge-coupled device) imaging system (Surface and Electro-Optics), it was observed that as the ratio of fp-151 increases, contact angle decreases, thus confirming that hydrophilicity increases, which are shown in FIG. 9.

3-2. Fourier-Transform Infrared Spectroscopy (FT-IR) Analysis

To confirm exposure of fp-151 on the surface of nanofiber, Fourier-transform infrared spectroscopy analysis was conducted using Nicolet 6700 spectrophotometer (Thermo). As the result, it was confirmed that as the ratio of fp-151 increases, peaks become larger at 1650 cm⁻¹ (amide I), 1540 cm⁻¹ (amide II), which cannot be seen in PCL nanofiber. Thus, by detecting peptide bond in protein through Fourier-transform infrared spectroscopy, it can be inferred that fp-151 protein is exposed on the surface of nanofiber, which are shown in FIG. 10.

3-3. X-Ray Photoelectron Spectroscopy (XPS) Analysis

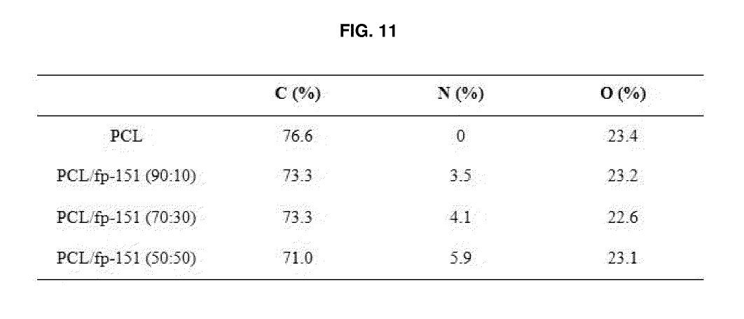

To confirm whether fp-151 is well exposed on the surface of nanofiber, X-ray photoelectron spectroscopy analysis was conducted using ESCALAB 2201XL (VG Scientific). As results of analyzing carbon (C), nitrogen (N), oxygen (O) contents, it was confirmed that as the ratio of fp-151 increases, nitrogen content which cannot be seen in PCL nanofiber increase more and more, which are shown in FIG. 11. And thus, from the detection of nitrogen existing in protein, it can be inferred that fp-151 protein is exposed on the surface of nanofiber.

3-4. Measurement of Mechanical Properties

Mechanical properties of the nanofibers prepared by blending PCL and fp-151 were measured. PCL, PCL/fp-151 (90:10), PCL/fp-151 (70:30), PCL/fp-151 (50:50) nanofibers were respectively cut to a size of 10 mm×25 mm, mechanical properties were measured while pulling them at a speed of 10 mm/min using universal testing machine (INSTRON) 10 N load cell. As the results, it was confirmed that as the ratio of fp-151 increases, elongation decreases more and more, but nanofiber containing fp-151 exhibits higher tensile strength and Young's modulus, compared to PCL nanofiber. Particularly, PCL/fp-151 (90:10) exhibited maximum tensile strength, and the value increased about 4 times compared to PCL, which are shown in FIGS. 12 and 13.

### Example 4

### Cell Culture on the Surface of Mussel Adhesive Protein-Based nanofiber

4-1. Preparation of PCL/fp-151-RGD Nanofiber

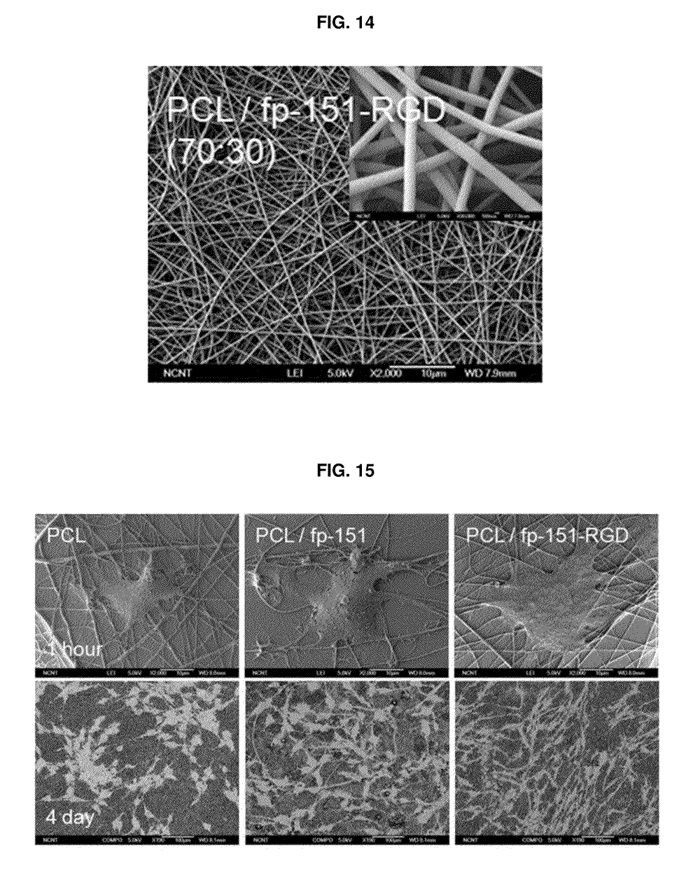

To effectively induce interactions of nanofiber and cells, nanofiber was prepared by the same method as <Example 2-1> using fp-151-RGD protein (SEQ ID NO. 2) wherein cell-recognition motif GRGDSP peptide is bonded to fp-151 C-terminal. As can be seen from the image analyzed by electron microscope shown in FIG. 14, it was confirmed that nanofiber using fp-151-RGD was also successfully prepared at a ratio of PCL and fp-151-RGD of 70:30 (w/w).

4-2. Osteoblast Shape and Population Analysis on PCL/fp-151-RGD Nanofiber

Cell culture experiment was conducted on PCL, PCL/fp-151, PCL/fp-151-RGD nanofibers. For experiment, mouse ostoeblastic MC3T3-E1 cell line was used, and the cells were cultured in a 5% CO₂ incubator using α-MEM medium containing 10% FBS (Fetal bovine serum) and 1× penicillin/streptomycin. All the cells were washed with PBS (phosphate buffered saline), ripped off with trypsin, and diluted in each medium that does not contain FBS to a concentration of 2×10⁵/ml. To analyze the shape of the cell, nanofiber was fixed on the bottom of the cell culture dish, and cells were introduced in the number of 3×10⁴ per nanofiber and cultured in a medium that does not contain FBS for 1 hour. To qualitatively analyze total population of the cells, the cells of the same number were cultured in a medium containing FBS for 4 days. To analyze electron microscope image, each sample was fixed with 2.5% glutaraldehyde for 30 minutes and dried under vacuum, and then, coated with platinum, and observed with electron microscope.

As the result, as shown in FIG. 15, it was confirmed that cell spreading is improved on PCL/fp-151 and PCL-fp-151-RGD nanofibers, compared to PCL nanofiber, and that the cells grow well along the fiber. It was also confirmed that total population of the cells increases even after culture for 4 days. This phenomenon was more improved on PCL/fp-151-RGD nanofiber.

4-3. Analysis of Attachment and Growth Degrees of Osteoblast on PCL/fp-151-RGD Nanofiber

To quantitatively analyze attachment and growth degrees of the cells, on the same nanofiber as <Example 4-2>, 3×10⁴ cells per nanofiber were cultured in a medium containing FBS for 1 day and 4 days, and then, the cells were ripped off with trypsin, and the number of the cells was measured with hemocytometer through trypan blue staining. As the result, it was confirmed that cell attachment and growth degrees improved more on PCL/fp-151 and PCL/fp-151-RGD nanofibers, compared to PCL nanofiber, and improved most on PCL/fp-151-RGD.

### Example 5

### Mounting of Various Functional Bioactive Materials on the Surface of Mussel Adhesive Protein-Based Nanofiber

5-1. Mounting of Functional Protein on PCL/fp-151 Nanofiber

To confirm whether functional protein may be conveniently mounted on the surface of nanofiber mixed with mussel adhesive protein without physical/chemical surface treatment, coating experiment was conducted. PCL nanofiber and PCL/fp-151 (70:30) nanofiber prepared by the method of <Example 2-1> were immersed in a solution where green fluorescent protein (GPF) is dissolved in the concentration of 1 mg/ml at 4° C. for 12 hours, washed with water three times using a shaker of 220 rpm, and then, the degree of coating of the remaining material was confirmed by fluorescence microscope. As the result, it was confirmed that green fluorescent protein is uniformly coated along the fiber only on PCL/fp-151 nanofiber, which is shown in FIG. 17.

5-2. Mounting of Functional Nucleic Acid on PCL/fp-151 Nanofiber

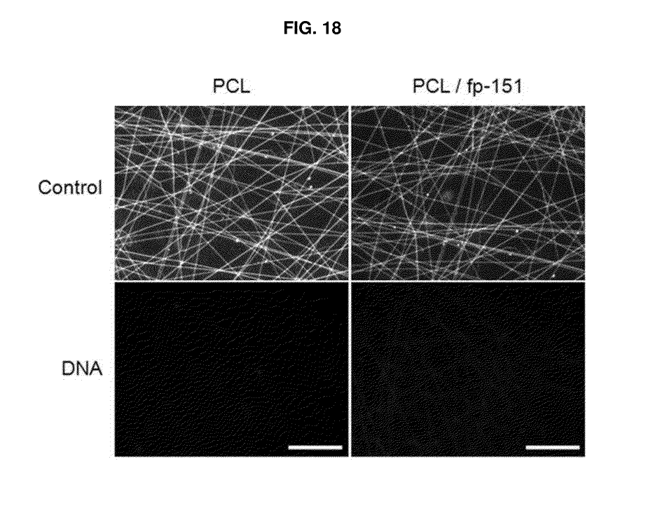

To confirm whether functional nucleic acid may be conveniently mounted on the surface of nanofiber mixed with mussel adhesive protein without physical/chemical surface treatment, coating experiment was conducted. PCL nanofiber and PCL/fp-151 nanofiber prepared by the method of <Example 2-1> were coated with a solution where plasmid nucleic acid is dissolved in the concentration of 20 ug/ml by the same method of <Example 5-1>, and the degree of coating of the remaining material was confirmed by fluorescence microscope after 4′,6-diamidino-2-phenylindole (DAPI) staining. As the result, it was confirmed that plasmid nucleic acid is uniformly coated along the fiber only on PCL/fp-151 nanofiber, which is shown in FIG. 18.

5-3. Mounting of Functional Saccharide on PCL/fp-151 Nanofiber

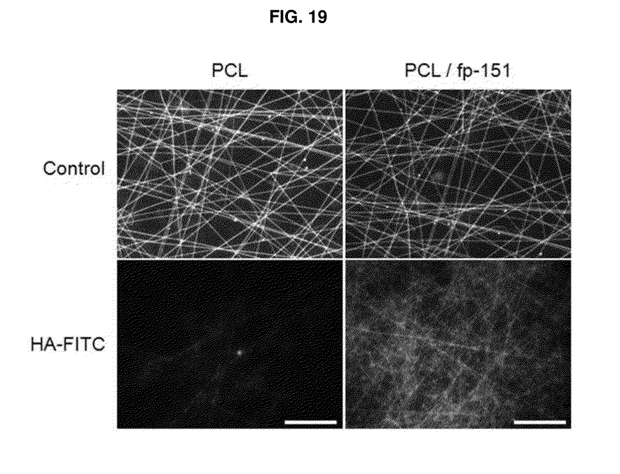

To confirm whether various functional saccharides may be conveniently mounted on the surface of nanofiber mixed with mussel adhesive protein without physical/chemical surface treatment, coating experiment was conducted. PCL nanofiber and PCL/fp-151 (70:30) nanofiber prepared by the method of <Example 2-1> were coated in a solution where fluorescein isothiocyanate-bonded hyaluronic acid (HA-FITC) is dissolved in the concentration of 1 mg/ml by the same method as <Example 5-1>, and the degree of coating of the remaining material was confirmed by fluorescence microscope. As the result, it was confirmed that hyaluronic acid is uniformly coated along the fiber only on PCL/fp-151 nanofiber, which is shown in FIG. 19.

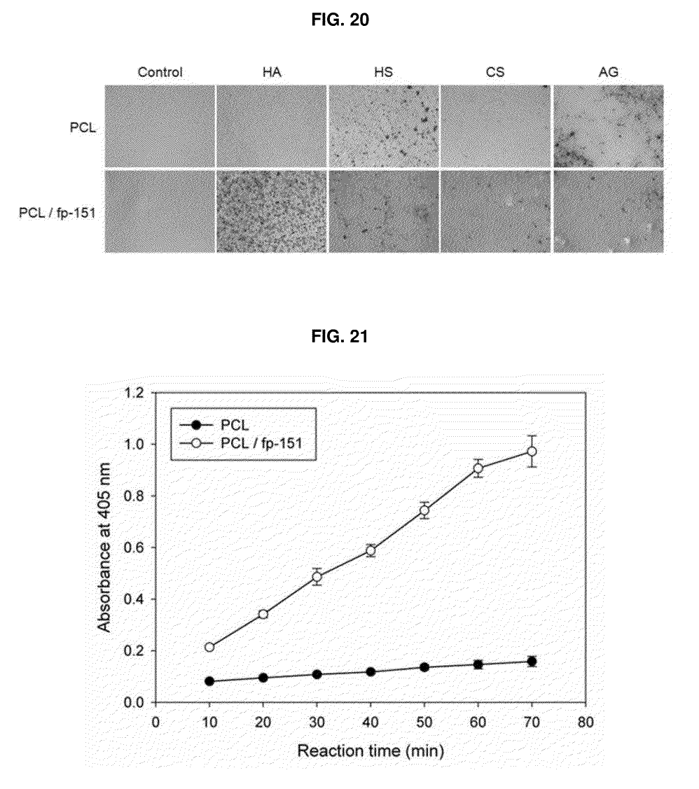

And, to confirm whether various functional saccharides in addition to hyaluronic acid may be bonded, coating experiment was conducted for extracellular matrix-derived saccharides of heparan sulfate (HS), chondroitin sulfate (CS), and natural saccharide of alginate (AG). The nanofiber was coated with a solution where heparan sulfate, chondroitin sulfate, and alginate are dissolved respectively in the concentration of 5 mg/ml by the same method as <Example 5-1>, washed, and immersed in 1% alcian blue solution (pH 2.5) to stain for 15 minutes, and then, the degree of coating was confirmed by optical microscope. As the result, it was confirmed that the degree of coating of various sacchrides is improved on PCL/fp-151 nanofiber, compared to PCL nanofiber, which is shown in FIG. 20.

5-4. Mounting of Functional Enzyme on PCL/fp-151 Nanofiber

To confirm whether functional enzyme may be conveniently mounted on the surface of nanofiber mixed with mussel adhesive protein while maintaining the activity without physical chemical surface treatment, PCL nanofiber and PCL/fp-151 (70:30) nanofiber prepared by the method of <Example 2-1> were coated with a solution where alkaline phosphatase is dissolved in the concentration of 100 ug/ml by the same method as <Example 5-1>, and washed, and then, the degree of coating of the remaining enzyme was examined by measuring the activity. The activity of the enzyme was confirmed by measuring absorbance at 405 nm to confirm conversion degree of the substrate pNPP (p-Nitrophenyl phosphate) using Alkaline Phosphatase Assay Kit (Anaspec). As the result, it was confirmed that alkaline phosphatase is coated only on PCL/fp-151 nanofiber to exhibit the activity, which is shown in FIG. 21.

5-5. Mounting of Functional Antibody on PVA/fp-151 Nanofiber

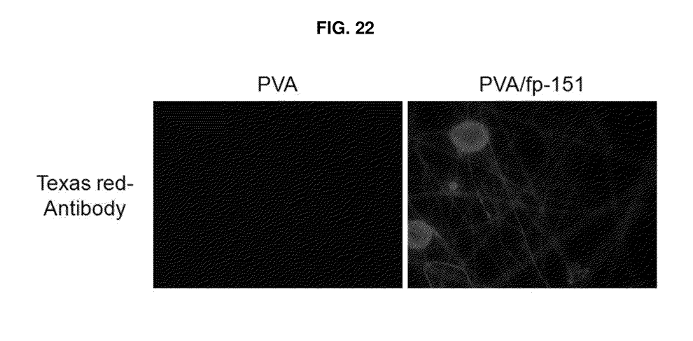

To confirm whether functional antibody may be conveniently mounted on the surface of nanofiber mixed with mussel adhesive protein without physical chemical surface treatment, coating experiment was conducted. First, PVA nanofiber and PVA/fp-151 (70:30) nanofiber were prepared by the method of <Example 2-1>, and then, cross-linking reaction was progressed for 12 hours using 50 mM glutaraldehyde vapor so as not to be dissolved in water. And then, the cross-linked nanofiber was coated with a solution where fluorescent material Texas Red-bonded antibody is dissolved in the concentration of 5 μg/ml by the same method as <Example 5-1>, and washed, and then, the degree of coating of the antibody was examined by fluorescence microscope. As the result, it was confirmed that the antibody is uniformly coated along the fiber only on PVA/fp-151 nanofiber, which is shown in FIG. 22.

What is claimed is:

1. A scaffold for tissue engineering, comprising mussel adhesive protein and bioactive substances attached to the mussel adhesive protein.

2. The scaffold according to claim 1, wherein the scaffold for tissue engineering comprises nanofiber comprising mussel adhesive protein or mussel adhesive protein and biodegradable polymer, and bioactive substances coated on the surface of the nanofiber.

3. The scaffold according to claim 1, comprising a metal surface coated with the mussel adhesive protein, and bioactive substances coated on the metal surface.

4. The scaffold according to claim 1, wherein the mussel adhesive protein is protein consisting of amino acid sequence set forth in SEQ ID NO. 4, protein consisting of amino acid sequence set forth in SEQ ID NO. 5, or protein consisting of 1 to 10 times consecutively connected amino acid sequence set forth in SEQ ID NO. 6.

5. The scaffold according to claim 1, wherein the mussel adhesive protein is fusion protein of at least two kinds selected from the group consisting of protein consisting of amino acid sequence set forth in SEQ ID NO. 4, protein consisting of amino acid sequence set forth in SEQ ID NO. 5, and protein consisting of 1 to 10 times consecutively connected amino acid sequence set forth in SEQ ID NO. 6.

6. The scaffold according to claim 5, wherein the mussel adhesive protein is protein consisting of amino acid sequence set forth in SEQ ID NO. 1 or protein consisting of amino acid sequence set forth in SEQ ID NO. 3.

7. The scaffold according to claim 1, wherein the mussel adhesive protein comprises polypeptide consisting of 3 to 25 amino acids comprising RGD (Arg Gly Asp), connected to the C-terminal or N-terminal.

8. The scaffold according to claim 7, wherein the polypeptide comprising RGD consists of amino acid sequence selected from the group consisting of SEQ ID NO. 8 to SEQ ID NO. 17.

9. The scaffold according to claim 7, wherein the mussel adhesive protein consists of amino acid sequence set forth in SEQ ID NO. 2.

10. The scaffold according to claim 1, wherein the bioactive substance is cell, protein, nucleic acid, fatty acid, carbohydrate, enzyme or antibody.

11. The scaffold according to claim 10, wherein the bioactive substance is selected from the group consisting of osteoblast, fibroblast, hepatocyte, neuron, cancer cell, B cell, white blood cell, stem cell, hyaluronic acid, heparan sulfate, chondroitin sulfate, alginate, dermatan sulfate, alkaline phosphatase, DNA, RNA, stem cell factor (SCF), vascular endothelial growth factor (VEGF), transforming growth factor (TGF), fibroblast growth factor (FGF), epidermal growth factor (EGF), cartilage growth factor (CGF), nerve growth factor (NGF), keratinocyte growth factor (KGF), skeletal growth factor (SGF), osteoblast-derived growth factor (BDGF), hepatocyte growth factor (HGF), insulin-like growth factor (IGF), cytokine growth factor (CGF), platelet-derived growth factor (PDGF), epithelial growth factor (EGF), bone growth factor, placental growth factor (PlGF), heparin-binding epidermal growth factor (HB-EGF), endothelial cell growth supplement (EGGS), colony stimulating factor (CSF), granulocyte macrophage-colony stimulating factor (GM-CSF), granulocyte colony stimulating factor (G-CSF), growth differentiation factor (GDF), integrin modulating factor (IMF), calmodulin (CaM), thymidinc kinase (TK), tumor necrosis factor (TNF), growth hormones (GH), growth hormone releasing hormone, growth hormone releasing peptide, glucagon-like peptides, G-protein-coupled receptor, macrophage activating factor, erythropoietin, macrophage peptide, B cell factor, T cell factor, protein A, allergy inhibitor, immunotoxin, lymphotoxin, tumor necrosis factor, tumor suppressors, metastasis growth factor, alpha-1 antitrypsin, albumin, alpha-lactalbumin, apolipoprotein-E, angiopoietins, hemoglobin, thrombin, thrombin receptor activating peptide, thrombomodulin, factor VII, factor VIIa, factor VIII, factor IX, factor XIII, plasminogen activating factor, fibrin-binding peptide, urokinase, streptokinase, hirudin, protein C, C-reactive protein, renin inhibitor, collagenase inhibitor, superoxide dismutase, leptin, angiostatin, angiotensin, bone stimulating protein, calcitonin, insulin, atriopeptin, cartilage inducing factor, elcatonin, connective tissue activating factor, tissue factor pathway inhibitor, follicle stimulating hormone, luteinizing hormone, luteinizing hormone releasing hormone, parathyroid hormone, relaxin, secretin, somatomedin, adrenocortical hormone, glucagon, cholecystokinin, pancreatic polypeptide, gastrin releasing peptide, corticotropin releasing factor, thyroid stimulating hormone, autotaxin, lactoferrin, myostatin, receptors, receptor antagonists, cell surface antigens, virus derived vaccine antigens, bone morphogenetic proteins (BMP), matrix metalloproteinase (MMP), tissue inhibitor matrix metalloproteinase (TIMP), interferons, interferon receptors, interleukins, interleukin receptors, interleukin binding proteins, cytokines, cytokine binding proteins, integrins, selectins, cadherins, collagen, elastin, lectins, fibrillins, nectins, fibronectin, vitronectin, hemonectin, laminin, glycosaminoglycans, hemonectin, thrombospondin, heparan sulfate, vitronectin, proteoglycans, transferrin, cytotactin, tenascin, lymphokines, neural cell adhesion molecules (N-CAMS), intercellular cell adhesion molecules (ICAMS), vascular cell adhesion molecule (VCAM), platelet-endothelial cell adhesion molecule (PECAM), monoclonal antibodies, polyclonal antibodies, antibody fragments, and combinations thereof.

12. The scaffold according to claim 2, wherein the biodegradable polymer is PCL (polycaprolactone), PDO (polydioxanone), PLLA (poly(L-lactide)), PLGA (poly(DL-lactide-co-glycolide)), PEO (polyethylene oxide) or PVA (polyvinyl alcohol).

13. The scaffold for tissue engineering according to claim 1, wherein the scaffold for tissue engineering is used for cell culture or tissue regeneration.

14. A method for manufacturing the scaffold for tissue engineering of claim 1, comprising
(1) preparing a nanofiber scaffold from mussel adhesive protein alone, or by mixing mussel adhesive protein and biodegradable polymer; and
(2) coating bioactive substance on the surface of the nanofiber scaffold.

15. A method for manufacturing the scaffold for tissue engineering of claim 1, comprising
(1) coating mussel adhesive protein on a metal surface; and
(2) coating bioactive substance on the metal surface.
